(19) United States
(12) Patent Application Publication
(10) Pub. No.: US 2016/0235501 A1
(43) Pub. Date: Aug. 18, 2016

(54) DEVICE WITH TROUGH TO CLEAN ELECTRODES USED FOR CAUTERIZATION

(71) Applicant: David Martini, Lincoln University, PA (US)

(72) Inventor: David Martini, Lincoln University, PA (US)

(21) Appl. No.: 15/044,623

(22) Filed: Feb. 16, 2016

Related U.S. Application Data
(60) Provisional application No. 62/116,615, filed on Feb. 16, 2015.

(51) Int. Cl. A61B 90/70; A61B 18/14

(52) U.S. Cl. CPC A61B 90/70; A61B 18/14; A61B 18/1442; A61B 2018/00976

(57) ABSTRACT

A cleaning device to remove waste material from a cauterization electrode including a main body, at least one receiving portion formed in the main body and configured to receive an electrode of a medical cauterization device, and a cleaning surface formed at least partially in the at least one receiving portion, and configured to remove debris from the electrode when contacted by the electrode.

### CROSS-REFERENCE TO RELATED APPLICATIONS

This application claims priority from U.S. Provisional Application No. 62/116,615, filed on Feb. 16, 2015.

### FIELD OF INVENTION

The present general inventive concept relates generally an electrode cleaning device, and, more particularly, to a cleaning device to allow a user to conveniently clean waste material from the electrodes of a medical apparatus.

### BACKGROUND

A cauterization device including one or more electrodes is used during surgery to minimize bleeding by fusing vessels and tissue. During use of the cauterization device in this manner, undesired materials and charring can build up on the electrodes. This can result in a decreased effectiveness of the cauterization device until the electrodes are cleaned.

### BRIEF SUMMARY

The present general inventive concept provides a cleaning device to remove waste material from a cauterization electrode of a medical device.

Additional aspects and advantages of the present general inventive concept will be set forth in part in the description which follows, and, in part, will be obvious from the description, or may be learned by practice of the present general inventive concept.

The foregoing and/or other aspects and advantages of the present general inventive concept may be achieved by a cleaning device to remove waste material from a cauterization electrode including a main body, at least one receiving portion formed in the main body and configured to receive an electrode of a medical cauterization device, and a cleaning surface formed at least partially in the at least one receiving portion, and configured to remove debris from the electrode when contacted by the electrode.

The foregoing and/or other aspects and advantages of the present general inventive concept may also be achieved by a cauterization electrode cleaning system includes a cauterization device having at least one electrode, and a cleaning member including a main body, at least one receiving portion formed in the main body and configured to receive the at least one electrode, and a cleaning surface formed at least partially in the at least one receiving portion, and configured to remove debris from the at least one electrode when contacted by the at least one electrode.

Other features and aspects may be apparent from the following detailed description, the drawings, and the claims.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1 illustrates a perspective view of an example bipolar cauterization device;

FIG. 2 illustrates a front view of a cleaning device according to an example embodiment of the present general inventive concept;

FIGS. 3-4 illustrate perspective views of an example system including a bipolar cauterization device and a cleaning device according to an example embodiment of the present general inventive concept; and

FIG. 5 illustrates another perspective view of the example system illustrated in FIGS. 3-4.

### DETAILED DESCRIPTION

Reference will now be made to various example embodiments of the present general inventive concept, examples of which are illustrated in the accompanying drawings and illustrations. The example embodiments are described herein in order to explain the present general inventive concept by referring to the figures.

The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. The described progression of processing operations described are merely examples, however, and the sequence of operations is not limited to that set forth herein and may be changed as is known in the art, with the exception of operations necessarily occurring in a certain order. Also, description of well-known functions and constructions may be omitted for increased clarity and conciseness.

Note that spatially relative terms, such as “up,” “down,” “right,” “left,” “beneath,” “below,” “lower,” “above,” “upper” and the like, may be used herein for ease of description to describe one element or feature's relationship to another element(s) or feature(s) as illustrated in the figures. Spatially relative terms are intended to encompass different orientations of the device in use or operation in addition to the orientation illustrated in the figures. For example, if the device in the figures is turned over or rotated, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

Various example embodiments of the present general inventive concept, as described herein, provide a cleaning device configured to remove waste material from one or more electrodes of a medical device. The cleaning device may be a component of a cleaning system which may include the medical device having the one or more electrodes.

In examples described herein, systems and methods are provided for cleaning the electrode tips of cauterization devices.

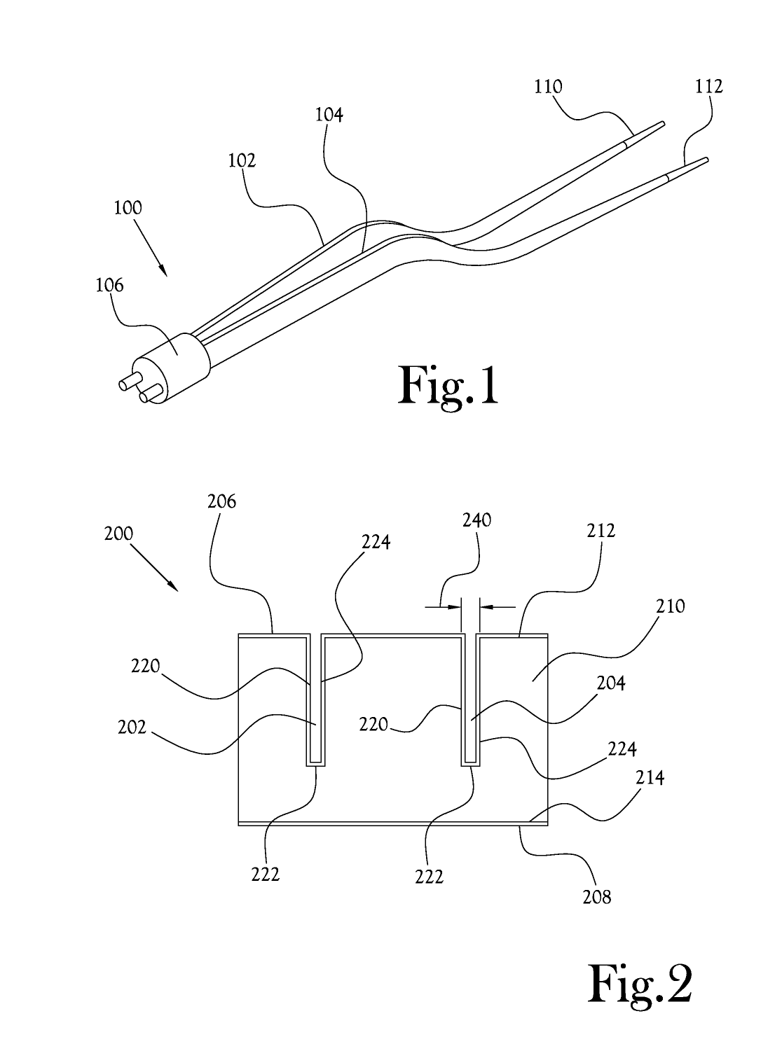

Referring now to FIG. 1, an example bipolar cauterization device 100 is illustrated. In this example, the bipolar cauterization device 100, sometimes referred to as bipolar forceps, includes a cap 106 that is coupled to a source of electricity during use. Two insulated arms 102, 104 extend from the cap 106, and electrodes 110, 112 are positioned at the ends of the two insulated arms 102, 104 respectively.

In use, the electrodes 110, 112 are positioned at desired locations on the body (e.g., at or near tissue and/or vessels), and electrical current is delivered to the electrodes 110, 112. This allows the bipolar cauterization device 100 to cauterize the tissue and/or vessels on the body.

During use, undesired materials can build up on the electrodes 110, 112. For example, debris from the patient can adhere to the electrodes 110, 112. Further, the current flowing through the electrodes 110, 112 can cause charring of the electrodes 110, 112. These undesired materials, which may be referred to herein as waste or waste material, can adversely impact the performance of the bipolar cauterization device 100.

FIG. 2 illustrates a front view of a cleaning device according to an example embodiment of the present general inventive concept. The example cleaning device 200 includes a body 210 that forms two grooves or troughs 202, 204. As described further, sides 220, 222, 224 that form the troughs 202, 204 are sized to receive the electrodes 110, 112, of the bipolar cauterization device 100 during cleaning. The troughs 202, 204 form a three-dimensional surface that is used to clean the bipolar cauterization device 100.

Portions of the body 210 include a cleaning surface 206 positioned thereon. In the example embodiment illustrated in FIG. 2, the cleaning surface 206 is provided at a top portion 212 of the body 210 and within the troughs 202, 204. In other example embodiments, the cleaning surface 206 can be positioned differently, or multiple cleaning surfaces can be provided.

According to various example embodiments, the cleaning surface 206 can be an abrasive surface, such as nylon, mineral particles, or other similar materials, that can be used to clean undesired debris from the electrodes 110, 112 as the electrodes 110, 112 are swiped or rubbed against the cleaning surface 206 as described herein. For example, the electrodes can be cleaned by sweeping them along the cleaning surface 206 of the top portion 212 and/or within the troughs 202, 204.

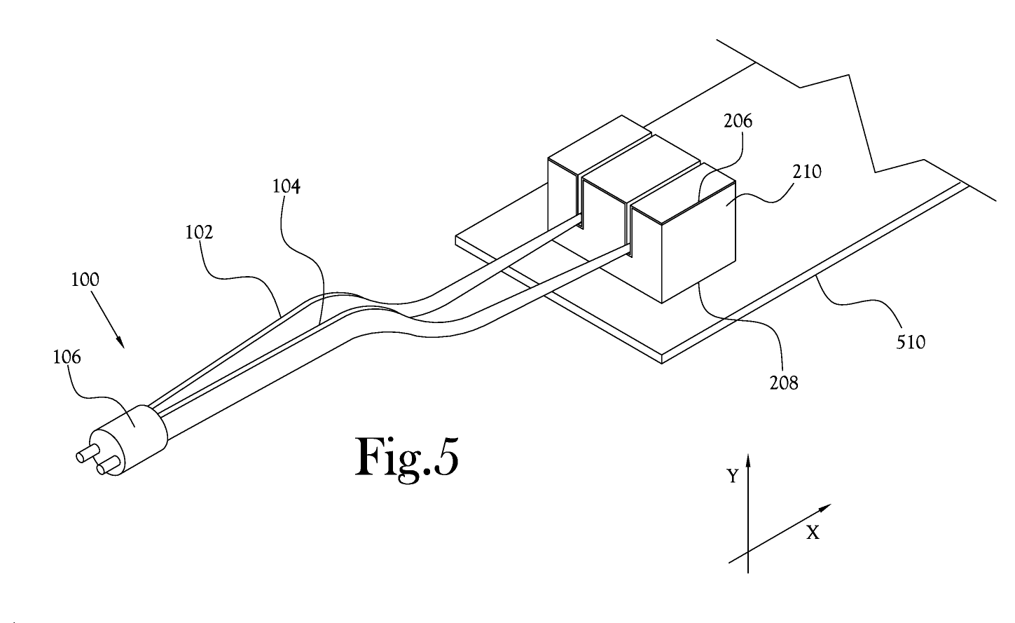

A bottom portion 214 of the body 210 includes an adhesive layer 208 that is used to affix the cleaning device 200 to a desired surface, such as a surface of a table 400 (see FIG. 5) located in a room in which a procedure is performed. In this example, the adhesive layer 208 is double-sided tape configured to affix one side to the body 210 and the other side to a desired surface onto which the cleaning device 200 is to be affixed, such as a surgical drape or other area of the surgical field.

In other example embodiments of the present general inventive concept, the cleaning device 200 can be affixed using other methods, such as by fastening devices like Velcro or screws. In yet other examples, the caregiver can simply hold the cleaning device 200 during use.

In this example embodiment, the body 210 of the cleaning device 200 is made of a flexible material, such as foam. This allows the body 210 to give slightly during use. In other examples, materials such as rubber or other plastics can be used. In yet other examples, more rigid materials can be used.

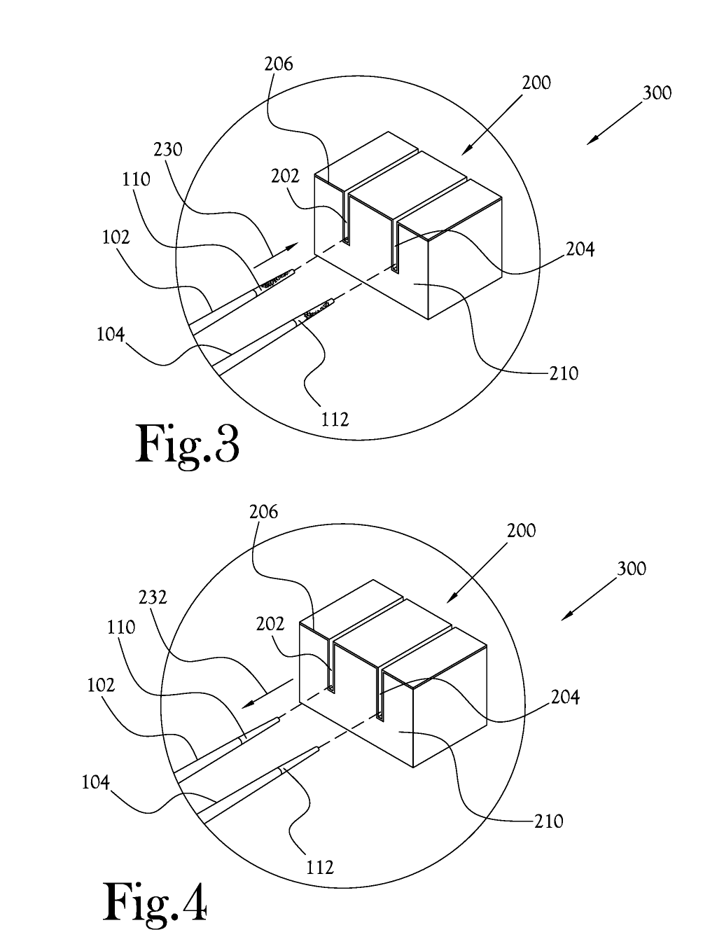

Referring now to FIGS. 3-4, an example system 300 showing the cleaning device 200 in use is provided. In this system 300, the user introduces the electrodes 110, 112 of the bipolar cauterization device 100, shown with waste material deposited thereon in FIG. 3, into the troughs 202, 204 of the cleaning device 200. The sides 220, 222, 224 forming each of the troughs 202, 204 are lined with the cleaning surface 206 so that the electrodes 110, 112 are cleaned by the cleaning surface 206 as the electrodes 110, 112 are introduced into and out of the troughs 202, 204 in any of up, down, forward, and backward directions (such as indicated by the x and y axis illustrated in FIG. 5). In addition, the electrodes 110, 112 can be rubbed or swiped onto the cleaning surface 206 located at the top portion 212 to further clean the electrodes 110, 112. Although the cleaning surface 206 is illustrated as covering all of the top surface and trough surfaces in this example embodiment, different example embodiments may include the cleaning surface 206 in less or more surface areas of the cleaning device 200.

In this example, a width 240 of each of the troughs 202, 204 (see FIG. 2) is sized so that the electrodes 110, 112 slidingly engage the cleaning surface 206 on each of the sides 220, 224 and/or side 222 (which may be referred to as the bottom of the troughs 202,204) as the electrodes 110, 112 are introduced into and out of the troughs 202, 204. The user can continue to rub the electrodes 110, 112 against the cleaning surface 206 until the desired debris is removed from the electrodes 110, 112. FIG. 5 illustrates the system 300 of FIGS. 3-4 with the electrodes 110,112 of the bipolar cauterization device 100 inserted into the troughs 202,204. FIG. 5 also illustrates the body 210 of the cleaning device 200 adhered to a surface 510 by the adhesive layer 208, so that a user may clean the electrodes 110,112 with a one-handed operation.

Over time, the cleaning surface 206 can deteriorate. As this occurs, the cleaning surface 206 can be cleaned (e.g., by removing debris). Once the useful life of the cleaning surface 206 is complete, the cleaning device 200 is discarded and a new cleaning device 200 is used. In an alternative embodiment, the cleaning surface 206 can be removed and a new cleaning surface 206 can be applied to the body 210.

Although the examples provided herein illustrate a bipolar cauterization device, other configurations are possible. For example, the cleaning device can be used to clean a monopolar cauterization device. In such a configuration, the body of the cleaning device can include only a single trough or multiple troughs as needed.

Further, the shape of the troughs on the cleaning device can be modified. For example, in another example embodiment of the present general inventive concept, the troughs can be closed on top so that tunnels are formed in the body of the cleaning device. In this manner, cleaning surfaces are provided on all sides, including the top side of the closed trough. In yet another example, the troughs can form a tortuous path as the troughs extend through the body of the cleaning device. This may allow for greater cleaning efficiently as the electrodes are introduced into and removed from the cleaning device.

According to various embodiments of the present general inventive concept, a cleaning device is provided to remove waste material from a cauterization electrode, the cleaning device including a main body, at least one receiving portion formed in the main body and configured to receive an electrode of a medical cauterization device, and a cleaning surface formed at least partially in the at least one receiving portion, and configured to remove debris from the electrode when contacted by the electrode. The cleaning surface may be formed on at least one outer surface of the main body. The at least one receiving portion may be formed as a trough accessing a top and front surface of the main body. The trough may further access a back surface of the main body. The at least one receiving portion may be formed as a tunnel extending from a front surface of the main body. The tunnel may form a throughway from the front surface to a back surface of the main body. The tunnel may be formed in a non-linear path.

According to various example embodiments of the present general inventive concept, a cauterization electrode cleaning system includes a cauterization device having at least one electrode, and a cleaning member including a main body, at least one receiving portion formed in the main body and configured to receive the at least one electrode, and a cleaning surface formed at least partially in the at least one receiving portion, and configured to remove debris from the at least one electrode when contacted by the at least one electrode. The cleaning surface may be formed on at least one outer surface of the main body. The at least one receiving portion may be formed as a trough accessing a top and front surface of the main body. The trough may further access a back surface of the main body. The at least one receiving portion may be formed as a tunnel extending from a front surface of the main body. The tunnel may form a throughway from the front surface to a back surface of the main body. The cleaning member may include two receiving portions configured to respectively receive electrodes of a two pronged medical cauterization device. The main body may be formed of a rigid material so as to be more frictionally abrasive to the at least one electrode. The main body may be formed of a resiliently pliable material so as to allow a user to squeeze the main body onto the at least one electrode during a cleaning operation. The main body may be formed of a foam material. The system may further include an adhesive layer provided to a bottom surface of the main body such that the cleaning member may be selectively adhered to another body or surface. The adhesive layer may be provided with a peelable layer which may be selectively removed to expose the adhesive layer. The adhesive layer may be VELCRO®. The cauterization device may be bipolar forceps, and at least two receiving portions may be provided to the cleaning member and configured to respectively receive electrodes of the bipolar forceps.

It is noted that the simplified diagrams and drawings do not illustrate all the various connections and assemblies of the various components, however, those skilled in the art will understand how to implement such connections and assemblies, based on the illustrated components, figures, and descriptions provided herein, using sound engineering judgment.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

While the present general inventive concept has been illustrated by description of several example embodiments, it is not the intention of the applicant to restrict or in any way limit the scope of the inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings.

1. A system, comprising:
a cauterization device having at least one electrode; and
a cleaning member comprising:
a main body,
at least one receiving portion formed in the main body and configured to receive the at least one electrode, and
a cleaning surface formed at least partially in the at least one receiving portion, and configured to remove debris from the at least one electrode when contacted by the at least one electrode.

2. The system of claim 1, wherein the cleaning surface is formed on at least one outer surface of the main body.

3. The system of claim 1, wherein the at least one receiving portion is formed as a trough accessing a top and front surface of the main body.

4. The system of claim 3, wherein the trough further accesses a back surface of the main body.

5. The system of claim 1, wherein the at least one receiving portion is formed as a tunnel extending from a front surface of the main body.

6. The system of claim 5, wherein the tunnel forms a throughway from the front surface to a back surface of the main body.

7. The system of claim 1, wherein the cleaning member comprises two receiving portions configured to respectively receive electrodes of a two pronged medical cauterization device.

8. The system of claim 1, wherein the main body is formed of a rigid material so as to be more frictionally abrasive to the at least one electrode.

9. The system of claim 1, wherein the main body is formed of a resiliently pliable material so as to allow a user to squeeze the main body onto the at least one electrode during a cleaning operation.

10. The system of claim 9, wherein the main body is formed of a foam material.

11. The system of claim 1, further comprising an adhesive layer provided to a bottom surface of the main body such that the cleaning member may be selectively adhered to another body or surface.

12. The system of claim 11, wherein the adhesive layer is provided with a peelable layer which may be selectively removed to expose the adhesive layer.

13. The system of claim 11, wherein the adhesive layer is VELCRO®.

14. The system of claim 1, wherein the cauterization device is bipolar forceps, and at least two receiving portions are provided to the cleaning member and are configured to respectively receive electrodes of the bipolar forceps.

15. A cleaning device to remove waste material from a cauterization electrode, comprising:
a main body;
at least one receiving portion formed in the main body and configured to receive an electrode of a medical cauterization device; and
a cleaning surface formed at least partially in the at least one receiving portion, and configured to remove debris from the electrode when contacted by the electrode.

16. The device of claim 15, wherein the cleaning surface is formed on at least one outer surface of the main body.

17. The device of claim 15, wherein the at least one receiving portion is formed as a trough accessing a top and front surface of the main body.

18. The device of claim 17, wherein the trough further accesses a back surface of the main body.

19. The device of claim 18, wherein the at least one receiving portion is formed as a tunnel extending from a front surface of the main body.

20. The device of claim 19, wherein the tunnel forms a non-linear throughway from the front surface to a back surface of the main body.
